(19) United States
(12) Patent Application Publication
(10) Pub. No.: US 2016/0166777 A1
(43) Pub. Date: Jun. 16, 2016

(54) DEVICE, SYSTEM AND METHOD FOR DELIVERY OF A LONG-ACTING DRUG

(71) Applicant: INSULINE MEDICAL LTD., Petach-Tikva (IL)

(72) Inventors: Gabriel BITTON, Jerusalem (IL); Ron NAGAR, Tel Aviv (IL)

(21) Appl. No.: 14/902,837

(22) PCT Filed: Jul. 3, 2014
(86) PCT No.: PCT/IB14/02369

§ 371 Date: Jan. 4, 2016

Related U.S. Application Data
(60) Provisional application No. 61/842,968, filed on Jul. 4, 2013.

(51) Int. Cl. A61M 5/44; A61M 5/172; A61K 45/06; A61K 9/00; A61K 38/28

(52) U.S. Cl. CPC A61M 5/445; A61K 9/0019; A61K 9/0014; A61K 38/28; A61K 45/06; A61M 5/1723; A61M 2230/005; A61M 2230/201; A61M 2205/3379; A61M 2205/18

(57) ABSTRACT

Embodiments of the present disclosure present systems, methods and devices relate to regulating drug absorption in a body of a patient, including delivering a dose of long-acting drug at a delivery site of a patient, applying a treatment to a treatment area surrounding and including the delivery site. A substantial portion of the drug may reside in tissue adjacent the treatment area for an extended period of time and may include a drug depot. The treatment may be configured to modify the level of at least one property of at least a portion of the treatment area. The absorption rate of the long-acting drag from the drug depot changes according to the level of the property.

### CROSS-REFERENCE TO RELATED APPLICATIONS

This application claims priority to U.S. Provisional Patent Application No. 61/842,968, filed Jul. 4, 2013, and entitled “Device and Method for Drug Delivery On-Demand” the disclosure of which is incorporated herein by reference in its entirety.

### FIELD OF THE DISCLOSURE

Some embodiments of the present disclosure generally relate e delivery of drugs, and in some embodiments to the delivery of long-acting drugs.

### BACKGROUND

Drug injection by syringe, pen injectors and other devices are used regularly for subcutaneous injections of therapeutic fluids, drugs, proteins, and other compounds. Such delivery systems and methods are also used routinely for insulin delivery.

Diabetic patients may require insulin injection around the clock to maintain proper blood glucose levels. Two types of insulin drugs are usually used: the first, a long-acting insulin that provides the basal insulin rate needed for maintaining patient's blood glucose level within a desired range between meals and overnight. The second is a short-acting insulin, bolus injection (or a “rapid-acting insulin”) that provides an amount of insulin corresponding to a dose of carbohydrates consumed by the patient during meals. The combination of a long-acting insulin and a short-acting insulin is called “basal-bolus therapy” or “intensive insulin therapy.” This therapy is used by most diabetes mellitus type 1 subjects as well as by part of the diabetes mellitus type II population, which are on multiple daily insulin injection therapy. There is an additional large population of subjects, typically diabetes mellitus type II subjects, that only inject a single long-acting insulin once a day, which needs to last the whole day.

When injecting long-acting insulin, the concentration of the insulin in the blood generally starts to increase within a half an hour to 1-2 hours, and is typically constant for a period of about 24 hours. Examples of such commercially available long-acting insulin analogs are insulin glargine marketed under the trade name LANTUS®, Lente insulin marketed under the trade name HUMULIN® and insulin detemir marketed under the trade name LEVEMIR®. An older version of insulin used for basal therapy is, for example, NPH (Neutral Prolamine Hagedorn) insulin.

The mechanism controlling the temporal profiles of long-acting insulin is different for the each of the different types of insulin analogs. For example, insulin glargine is soluble at pH 4, while in neutral pH it forms precipitates. When injected into the subcutaneous tissue it resides there in the form of microprecipitates of multi-hexamer structures. The insulin glargine is slowly dissolved into single hexamers and then to dimmers at a rate which is dependent on the local pH level at the drug depot. The insulin glargine is dissolved into monomers and released from the drug depot to the blood system.

The long action of the insulin detemir results from the addition of fatty acid side chains to native insulin, which stabilizes its self-association into hexamers and permits reversible insulin-albumin binding. When insulin detemir is injected to the subcutaneous tissue it aggregates into hexamers at the drug depot. The insulin detemir slowly dissociates into dimers and monomers, which are then absorbed in the bloodstream. Once in the circulation, insulin detemir may be 98% albumin bound, which also contributes to its protracted action.

One of the main drawbacks of insulin therapy compared to normal physiology is its increased variability in terms of the pharmacokinetics and pharmacodynamics profile during the time the long-acting insulin is active in the patient, leading to an unpredictable effect of the drug. Additionally, variability may be caused by fluctuations in basal insulin pharmacokinetics and pharmacodynamics profiles, which can be inherent to the absorption process. The basal insulin can take a few hours to reach an insulin plateau, which following thereof, the insulin plateau can decrease towards the end of the lifetime of the basal insulin before receiving a new drug injection.

Variability may further be caused by the patient activity, such as eating, fasting and physical activity, and/or by environmental factors, such as the ambient temperature, for example.

Moreover, upon injecting the long-acting insulin repeatedly, such as day after day, there may be an accumulation of the drug and it takes about 2-4 days to reach a stable insulin concentration. Hence any interference in the long-acting insulin absorption at a given day, such as due to illness or failing to inject the long acting-insulin at a given day, may result in fluctuation of basal insulin concentration for several days afterwards.

The variability in the pharmacokinetics and pharmacodynamics profile may result in any one of the following: increased risk of hypoglycemia; increased weight gain associated with defensive eating to prevent hypoglycemia; changes in appetite due to fluctuations in glucose or insulin levels; reduced patient confidence in their treatment due to variability in the glucose levels; increased risk of development and/or progression of diabetes complications; and increased risk of mortality.

A large variation in the pharmacokinetics and pharmacodynamics profile of long-acting insulin analogs is known and can lead to, for example, hypoglycaemic events. See, e.g., Heise et al, “Insulin Degludee: Four Times Lower Pharmacodynamic Variability than Insulin Glargine Under Steady-State Conditions in Type 1 Diabetes” Diabetes Obes Metab. 2012 September; 14(9):859-64. doi: 10.1111/j.1463-1326.2012.01627.x. Epub 2012 Jun. 7); and Ratner et al., “Hypoglycaemia Risk with Insulin Degludee Compared with Insulin Glargine in Type II and Type I Diabetes: a Pre-Planned Meta-Analysis of Phase 3 Trials”, Diabetes Obes Metab. 2013 February; 15(2):175-84. doi: 10.1111/dom.12032. Epub 2012 Dec. 3.

FIG. 1 shows a 24 hour profile of insulin analogue concentration in the blood of subcutaneous insulin infusion (CSII) of lispro insulin, and injection of glargine Insulin (LANTUS), injection of NPH insulin and injection of Lente insulin. It can be seen that all drugs experienced changes in their concentration during the 24 hour period of monitoring, Even during the infusion of insulin, which has a flatter profile, there still is detected a passage of a relatively long time, until it reaches a substantially stable level.

FIG. 2 shows a 24 hour profile of injection of glargine Insulin (LANTUS) providing the basal insulin rate and injection of a short-acting insulin, bolus injection that provides an amount of insulin for matching a dose of carbohydrates consumed by the patient during breakfast, lunch and supper.

Hence there is a need to improve and reduce the insulin variability of long-acting insulin and the variability of long-acting drugs.

### SUMMARY OF DISCLOSURE

In some embodiments, the current subject matter relates to systems, methods and devices that can regulate the absorption of a long-acting drug in the body of a patient.

There is provided according to an embodiment of the present disclosure, a method for regulating the glucose level in a body of a patient, including injecting a dose of long-acting insulin at an injection site of a patient, applying a treatment to a treatment area surrounding and including the injection site. A substantial portion of the injected insulin may reside in tissue adjacent the treatment area for an extended period of time and may include an insulin depot. The treatment may be configured to modify the level of at least one property of at least a portion of the treatment area. The absorption rate of the long-acting insulin from the insulin depot changes according to the level of the property.

In some embodiments, the treatment may include at least one of: heating, cooling, suction, depression, massage, energy, radiation, mechanical vibration, electrical stimulation, acoustic stimulation, magnetic stimulation, electromagnetic stimulation, radio frequency irradiation, microwave irradiation, injection of an additional substance, application of a cream, Transcutaneous Electrical Nerve Stimulation (“TENS”), drugs, medicament, chemicals, biologically active bacteria, biologically inactive bacteria, an analgesic and a vasodilator.

In some embodiments, at least one property of the treatment area includes at least one of: temperature, pH, blood perfusion, chemical structure of the insulin, and oxygen saturation.

In some embodiments, a first treatment may be applied to modify a level of the blood perfusion and a second treatment may be applied to modify the chemical structure. The absorption rate of the long-acting insulin may be increased or decreased.

In some embodiments, the treatment includes heat, and the absorption rate increases when heat may be applied. In some embodiments, the treatment includes cooling, and the absorption rate decreases when cooling may be applied. In some embodiments, the treatment may be configured to modify the at least one property to effect an increase in absorption rate to mimic a bolus dose injection. In some embodiments, the treatment modifies the pH level of the treatment area. In some embodiments, the treatment may be configured to change the chemical structure of the long-acting insulin from at least any one of: a microprecipitated structure to a hexamer structure and/or a hexamer structure to a monomer structure.

In some embodiments, the dose includes sufficient long-acting insulin:
for at least an eight hour period for the patient;
for at least a twenty-hour hour period or more for the patient;
for at least a forty-eight hour period or more for the patient;
for at least a seventy-two hour period or more for the patient;
for at least a week or more for the patient;
for at least two weeks or more for the patient;
for at least a month or more for the patient;
for at least a few months or more for the patient; and/or
for at least a six months or more for the patient.

In some embodiments, the treatment may be applied:
during the first four hours of a twenty-four hour period after injection and the last four hours of the twenty-four hour period; and/or
intermittently during the first four hours and the last four hours, where the intermittent application of the treatment may be about ten minutes of treatment, followed by about ten minutes of no treatment.

In some embodiments, the treatment may be configured to modify the pH level of at least a portion of the treatment area. The pH level may be modified:
to a more acidic pH level;
to a pH level of between about 4 and about pH 5; and/or
to a pH level of about 4.6.

In some embodiments, the method may further include adjusting the amount of the treatment. The treatment may be adjusted based on an activity level of the patient. The treatment may be applied so as to modify the level of the at least one property so as to change the absorption rate to mimic a bolus dose injection.

In some embodiments, at least one property may include a pH level, and decreasing the pH of the insulin depot results in an increase in the absorption rate of the insulin from the insulin depot. In some embodiments, an increase in the absorption rate can be reduced by neutralizing the pH level. In some embodiments, neutralizing the pH level may include changing the pH level to between about 6.8 and about 7.7. In some embodiments, neutralizing the pH level includes changing the pH level to approximately the pH level of the body of the patient. In some embodiments. neutralizing the pH level includes changing the pH level to about 7.2.

In some embodiments, the pH level of the insulin depot may change by changing the temperature of the treatment area, and the treatment may include application of a cream to the treatment area and at least on property may be the pH level of the insulin depot.

In some embodiments, the treatment may be applied via the treatment element, where the treatment element may be placed on and/or around the treatment area, and where the treatment element may define the treatment area.

In some embodiments, the method may include activating the treatment element, which may be activated automatically or by the patient. The automatic activation may be based on a predetermined schedule.

In some embodiments, the method may include detecting when the injection has been made. The detecting may be determined by a sensor provided with a treatment element configured to apply the treatment. Detecting the blood glucose level of the patient may be via a blood glucose monitor provided with the treatment element.

In some embodiments, the treatment may be applied when the blood-glucose monitor detects a predetermined blood glucose level. In some embodiments, the treatment may be ceased when the blood-glucose monitor detects a predetermined glucose level. In some embodiments, the amount and/or type of treatment corresponds to a plurality of predetermined blood glucose levels.

In some embodiments, the treatment may include heating the treatment area when a first blood glucose level may be detected. The treatment may include cooling the treatment area when a second blood glucose level, lower than the first blood glucose level may be detected.

In some embodiments, the treatment may be configured to smooth at least one of the pharmacokinetic and pharmacodynamic profiles of the injected long-acting insulin.

In some embodiments, the treatment may be configured to achieve a substantially flat pharmacokinetic and/or pharmacodynamic profile of the injected long-acting insulin.

In some embodiments, blood perfusion may be monitored at or adjacent the insulin depot.

In some embodiments, the treatment may be applied when the blood perfusion is equal or lower than a predetermined threshold or the treatment may be stopped when the blood perfusion is equal to or greater than the predetermined threshold.

In some embodiments, a size of the insulin depot or a rate of change of the size of the insulin depot may be determined In some embodiments, the treatment may be applied dependent upon the determined rate of change in the size of the insulin depot.

In some embodiments, the treatment may be applied when the rate of change in the size of the insulin depot decreases. In some embodiments, the treatment may be stopped when the rate of change in the size of the insulin depot increases beyond a predetermined threshold. In some embodiments, a secondary treatment may be applied when the rate of change in the size of the insulin depot increases beyond a predetermined threshold. The size of the insulin depot may be determined via at least one of: electrical impedance, optically, by measuring micro-precipitates, by measuring a concentration of insulin molecules, and/or by measuring an amount of insulin molecules.

In some embodiments, application of the treatment may be activated by the patient so as to increase the absorption of the long-acting insulin from the insulin depot to mimic a bolus dose injection. The mimicked insulin bolus injection may achieve substantially the same pharmacokinetic and/or pharmacodynamics profile as a bolus dose injection.

In some embodiments of the present disclosure, a method for regulating the absorption of a long-acting drug in the body of a patient is presented and includes: receiving an indication of delivery of a long-acting drug into a drug depot of a patient, the drug depot including subcutaneous tissue adjacent and including a delivery site which retains a substantial amount of the delivered drug, determining, using at least one sensor, at least one statistic or measurement associated with at least one of the patient and the drug, determining, via a processor, at least one treatment to apply to the surface of the skin of the patient in an area surrounding and including the delivery site. At least one treatment may be configured to modify the absorption of the long-acting drug from the drug depot into the bloodstream of the patient based on the statistic or measurement, and applying the at least one treatment using a treatment element, wherein the application of at least one treatment element varies the absorption rate of the long-acting drug.

In some embodiments, the long-acting drug may be long-acting insulin.

At least one statistic or measurement may be at least one of: time since last injection, blood glucose level, local blood perfusion, absorption rate, size of a long-acting drug depot, rate of change of the size of the long-acting drug depot, pH of the treatment area, temperature of the treatment area. In some embodiments, the treatment may include at least one of: heating, cooling, suction, depression, massage, energy, radiation, mechanical vibration, electrical stimulation, acoustic stimulation, magnetic stimulation, electromagnetic stimulation, radio frequency irradiation, microwave irradiation, injection of an additional substance, application of a cream, Transcutaneous Electrical Nerve Stimulation (“TENS”), drugs, medicament, chemicals, biologically active bacteria, biologically inactive bacteria, an analgesic and a vasodilator.

In some embodiments, the varied absorption rate may create an improved pharmacokinetic and/or pharmacodynamics profile. In some embodiments, an amount of physical activity of the patient may be detected. Application of the at least one treatment may be varied based on the amount of physical activity of the patient.

In some embodiments, the application of at least one treatment may be an intermittent application of the at least one treatment. In some embodiments, the application of at least one treatment may be based on the amount of time since the indication of the injection was received.

In some embodiments, upon detection that an amount of the long-acting drug in the drug depot is below a predetermined threshold, an alert may be generated. The alert may be at least one of: sounding an acoustic noise, vibrating, lighting an indicator light, changing a color of the indicator light, and sending an alert to the patient via at least one of: an SMS message, a text message, an MMS message, an email, and a phone call. An intensity of the alert may change depending upon the amount of the long-acting drug in the drug depot.

In some embodiments of the present disclosure, a method for regulating absorption of a long-acting drug in the body of a patient is provided and includes: delivering a dose of a long-acting drug at a delivery site of a patient, applying a treatment to a treatment area surrounding and including the delivery site, wherein a substantial portion of the delivered drug resides in tissue adjacent the treatment area for an extended period of time and includes a drug depot. The treatment may be configured to modify the level of at least one property of at least a portion of the treatment area. The absorption rate of the long-acting drug from the drug depot may change according to the level of the property.

In some embodiments of the present disclosure, a system for regulating the absorption of a long-acting drug in the body of a patient is provided, and includes a treatment element configured to apply treatment to the surface of the skin surrounding and including a delivery site. In such embodiments, a dose of a long-acting drug may be delivered into subcutaneous tissue, and the subcutaneous tissue adjacent and including the injection site may include or comprise a long-acting drug depot for containing the injected long-acting drug for an extended period of time. The treatment element may be configured to apply treatment to modify the level of at least one property of the treatment area so as to effect a change in the absorption rate of the long-acting drug from the drug depot. The system may further include a processor having computer instructions operational thereon which may be configured for causing the processor to operate the treatment element to apply treatment.

In some embodiments of the present disclosure, a system for regulating the glucose level in a body of a patient is provided and includes a treatment element configured to apply treatment to the surface of e skin surrounding and including an injection site where a dose of a long-acting insulin is injected into subcutaneous tissue. The subcutaneous tissue adjacent and including the injection site may include or comprises a long-acting insulin depot for containing the injected long-acting insulin for an extended period of time. The treatment element may be configured to apply treatment to modify the level of at least one property of the treatment area so as to effect a change in the absorption rate of the long-acting insulin from the insulin depot. The system may further include a processor having computer instructions operational thereon which are configured for causing the processor to operate the treatment element to apply treatment.

In some embodiments, the treatment element may include at least one of: heater, a cooling device, a suction device, a transducer, a radiation delivery element, one or more electrodes, an injector, and a dispenser. In some embodiments, the treatment may include at least one of: heating, cooling, suction, depression, massage, energy, radiation, mechanical vibration, electrical stimulation, acoustic stimulation, magnetic stimulation, electromagnetic stimulation, radio frequency irradiation, microwave irradiation, injection of an additional substance, application of a cream, Transcutaneous Electrical Nerve Stimulation (“TENS”), drugs, medicament, chemicals, biologically active bacteria, biologically inactive bacteria, an analgesic and a vasodilator.

In some embodiments, at least one properly of the treatment area includes at least one of temperature, pH, blood perfusion, chemical structure of the insulin and oxygen saturation.

In some embodiments, the absorption rate of the long-acting insulin may be increased or decreased based upon the treatment applied by the treatment element.

The treatment element may include a heater, and wherein the computer instructions are configured to cause the processor to operate the heater to increase the absorption rate of the insulin from the drug depot. In some embodiments, the treatment applied by the treatment element may include cooling, and wherein the absorption rate decreases when cooling may be applied.

In some embodiments, the computer instructions may be additionally configured to cause the processor to operate the treatment element to modify the property to effect an increase in absorption rate to mimic a bolus dose injection.

In some embodiments, the computer instructions may be configured to cause the processor to operate the treatment element to modify the pH level of the treatment area. In some embodiments, the computer instructions may be configured to cause the processor to operate the treatment element to effect a change in the chemical structure of the long-acting insulin from at least any one of: a microprecipitated structure to a hexamer structure and/or a hexamer structure to a monomer structure.

In some embodiments, the dose may include sufficient long-acting for at least an eight hour period for the patient.

In some embodiments, the computer instructions moray be configured to cause the processor to operate the treatment element:
during the first four hours of the twenty-four hour period after injection and the last four hours of the twenty-four hour period;
intermittently during the first four hours and the last four hours; and/or
for about ten minutes of treatment, followed by about ten minutes of no treatment.

In some embodiments, the computer instructions may be additionally configured to cause the processor to operate the treatment element to:
modify the pH level of at least a portion of the treatment area;
modify the pH level to a more acidic pH level;
modify the pH level of between about 4 and about pH 5; and/or
modify the pH level to a pH level of about 4.6.

In some embodiments, the computer instructions may be configured to cause the processor to operate the treatment element to:
adjust the amount of the treatment;
based on an activity level of the patient; and
modify the level of the at least one property so as to change the absorption rate to mimic a bolus dose injection.

In some embodiments, at least one property includes pH, and the computer instructions may be configured for decreasing the pH of the insulin depot to effectuate an increase in the absorption rate of the insulin from the insulin depot, which may be accomplished by neutralizing the pH level.

In some embodiments, the computer instructions may be configured to cause the processor to operate the treatment element to:
effect neutralizing the pH level to a pH level to between about 6.8 and about 7.7;
effect neutralizing the pH level to a pH level corresponding to approximately that of the body of the patient;
effect neutralizing the pH level by effectuating a change in the pH level to about 7.2; and/or
neutralize the pH level by effectuating a change in the pH level of the insulin depot by effecting a change the temperature of the treatment area.

In some embodiments, the treatment applied by the treatment element includes application of a cream to the treatment area and wherein the at least on property may be pH level of the insulin depot, which may be accomplished via the application of a cream to the treatment area and wherein the at least on property may be pH level of the treatment area.

In some embodiments, the treatment may be applied via the treatment element. The treatment element may be configured to be placed on and/or around the tissue adjacent and including the injection site. The treatment element may be configured to define the treatment area. The treatment element may be activated by the patient or based on computer instructions configured to cause the processor to operate the treatment element to apply treatment.

In some embodiments, the computer instructions may be configured to cause the processor to operate the treatment element to apply treatment based on a predetermined schedule.

In some embodiments, a sensor to detect when an injection has been made may be provided. The sensor may be provided with the treatment element.

In some embodiments, a blood glucose monitor may be provided with the treatment element, wherein the blood glucose monitor may be configured to detect blood glucose level of the patient. The computer instructions may be configured to cause the processor to operate the treatment element to apply treatment when the blood-glucose monitor detects a pre-determined blood glucose level or to cause the processor to operate the treatment element to cease applying the treatment when the blood-glucose monitor detects the pre-determined glucose level. In some embodiments, the computer instructions may be configured to cause the processor to determine the amount and/or type of treatment to be applied, wherein the amount and/or type of treatment may correspond to a plurality of pre-determined blood glucose levels.

In some embodiments, the computer instructions may be configured to cause the processor to operate the treatment element to apply a first treatment to the treatment area when a first blood glucose level may be detected, wherein the first treatment includes at least heating the treatment area. The computer instructions may be configured to cause the processor to operate the treatment element to apply a second treatment to the treatment area when a second blood glucose level, lower than the first blood glucose level may be detected. The second treatment may include at least cooling the treatment area.

In some embodiments, the computer instructions may be configured to cause the processor to operate the treatment element to apply treatment configured to smooth at least one of the pharmacokinetic and pharmacodynamic profiles of the injected long-acting insulin.

The computer instructions may be configured to cause the processor to operate the treatment element to apply treatment configured to achieve a substantially flat pharmacokinetic and/or pharmacodynamic profile of the injected long-acting insulin.

In some embodiments, a sensor may monitor blood perfusion at or adjacent the insulin depot. To that end, in some embodiments, the computer instructions may be configured to cause the processor to operate the treatment element to apply treatment when the blood perfusion is equal or lower than a predetermined threshold or to cause the processor to operate the treatment element to stop treatment when the blood perfusion is equal to or greater than the predetermined threshold.

In some embodiments, a sensor may determine a size of the insulin depot or to determine a rate of change of the size of the insulin depot. In some embodiments, the computer instructions may be configured to cause the processor to operate the treatment element to apply treatment based upon the determined rate of change in the size of the insulin depot.

In some embodiments, the computer instructions may be configured for causing the processor to operate the treatment element to apply treatment when the rate of change in the size of the insulin depot decreases. The computer instructions may be configured for causing the processor to operate the treatment element to stop treatment when the rate of change in the size of the insulin depot increases beyond a predetermined threshold. The computer instructions may be configured for causing the processor to operate the treatment element to apply a secondary treatment when the rate of change in the size of the insulin depot increases beyond a predetermined threshold.

In some embodiments, the sensor may be configured to detect the size of the insulin depot via at least one of electrical impedance, optically, by measuring micro-precipitates, by measuring a concentration of insulin molecules, and by measuring an amount of insulin molecules.

In some embodiments, the sensor may be configured to detect the size of the insulin depot by measuring micro-precipitates, a concentration of insulin molecules, and an amount of insulin molecules. The computer instructions may be configured to cause the processor to operate the treatment element to apply treatment when the treatment may be activated by the patient so as to increase the absorption of the long-acting insulin from the insulin depot to is a bolus dose injection.

In some embodiments, the mimicked insulin bolus injection achieves substantially the same pharmacokinetic and/or pharmacodynamics profile as a bolus dose injection.

There is provided according to an embodiment of the present disclosure, a system for regulating glucose level in a body of a patient, including: a processor to receive an indication of delivery of a long-acting drug into a drug depot of a patient, the drug depot including subcutaneous tissue adjacent and including the delivery site which retains a substantial amount of the delivered drug, at least one sensor to detect at least one statistic or measurement associated with at least one of the patient and the drug. A processor having computer instructions operational thereon may be configured to cause the processor to determine at least one treatment to apply to the surface of the skin of the patient in an area surrounding and including the delivery site. At least one treatment may be configured to modify the absorption of the long-acting drug from the drug depot into the bloodstream of the patient based on the statistic or measurement, and a treatment element to apply the at least one treatment, wherein the application of the at least one treatment element varies the absorption rate of the long-acting drug.

In some embodiments, the long-acting drug may be long-acting insulin. At least one statistic or measurement may be at least one of: time since last injection, blood glucose level, local blood perfusion, absorption rate, size of a long-acting drug depot, rate of change of the size of the long-acting drug depot, pH of the treatment area, temperature of the treatment area. At least one treatment may be at least one of heat, cold, suction, depression, massage, energy, radiation, mechanical vibration, electrical stimulation, acoustic stimulation, magnetic stimulation, electromagnetic stimulation, radio frequency irradiation, microwave irradiation, injection of an additional substance, application of a cream, Transcutaneous Electrical Nerve Stimulation (“TENS”), drugs, medicament, chemicals, biologically active bacteria, biologically inactive bacteria, an analgesic and a vasodilator.

In some embodiments, the varied absorption rate creates an improved pharmacokinetic and/or pharmacodynamics profile. A sensor may detect an amount of physical activity of the patient, and the computer instructions may be configured to vary the application of the at least one treatment based on the amount of physical activity of the patient.

In some embodiments, the computer instructions may be configured to cause the processor to operate the treatment element to apply at least one treatment as an intermittent application of the at least one treatment.

The computer instructions may be configured to cause the processor to operate the treatment element to apply at least one treatment based on the amount of time since the indication of the injection was received.

The system may further include a sensor to detect that an amount of the long-acting drug in the drug depot is below a predetermined threshold, and an alerting mechanism to generate an alert.

The alert may be at least one of: sounding an acoustic noise, vibrating, lighting an indicator light, changing a color of the indicator light, and sending an alert to the patient via at least one of: an SMS message, a text message, an MMS message, an email, and a phone call.

An intensity of the alert may change depending upon the amount of the long-acting drug in the drug depot.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The principles and operations of the systems, apparatuses and methods according to some embodiments of the present disclosure may be better understood with reference to the drawings, and the following description. The drawings are given for illustrative purposes only and are not meant to be limiting.

FIG. 1 is a graph showing results of a study of the concentration profile of four different insulin analogues in the blood during a 24 hour period.

FIG. 2 is a graph showing results of a study of the concentration profile of combination of a long-acting insulin and a short-acting insulin analog in the blood during a 24 hour period.

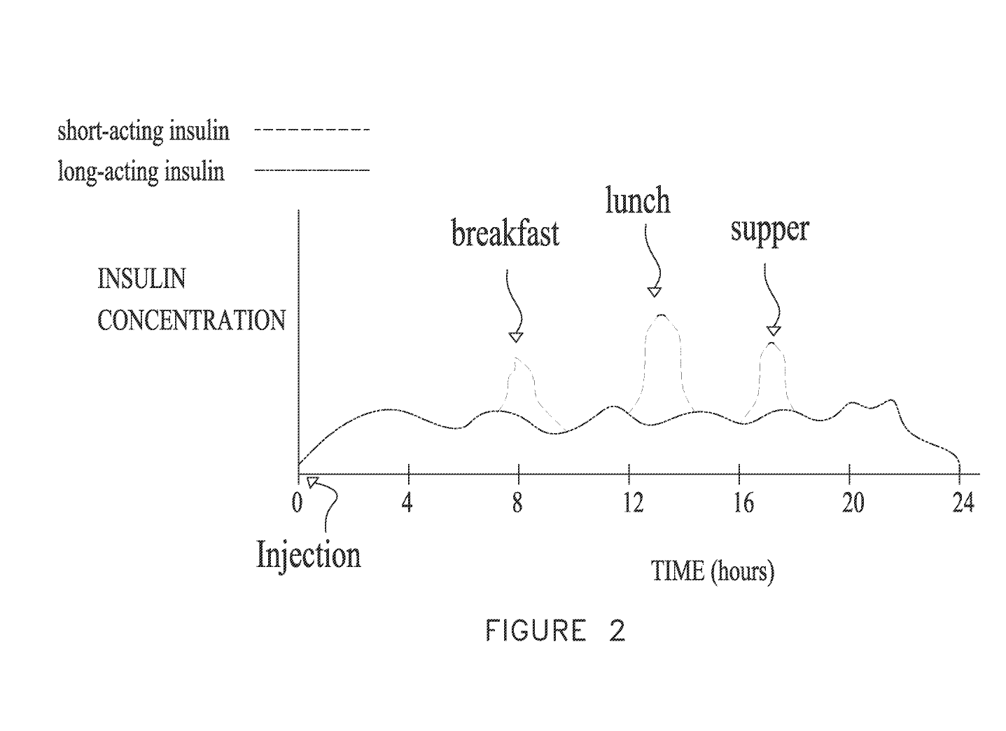

FIG. 3 is an illustration of an exemplary system for regulating the absorption of a drug, according to some embodiments of the present disclosure;

FIG. 4 is an illustration of an exemplary system for regulating the absorption of a drug, according to some embodiments of the present disclosure;

FIG. 5 is an illustration of an exemplary system for regulating the absorption of a drug, according to some embodiments of the present disclosure;

FIG. 6 is an illustration of an exemplary system for regulating the absorption of a drug, according to some embodiments of the present disclosure;

FIG. 7 is an illustration of an exemplary system for regulating the absorption of a drug, according to some embodiments of the present disclosure;

FIG. 8 is a graph showing a temporal concentration profile according to some embodiments of the present disclosure;

FIG. 9 is a graph showing a temporal concentration profile according to some embodiments of the present disclosure;

FIG. 10 is a graph showing a temporal concentration profile according to some embodiments of the present disclosure; and

FIG. 11 is an exemplary schematic flow chart of a system and method for regulating the absorption of a drug.

### DETAILED DESCRIPTION

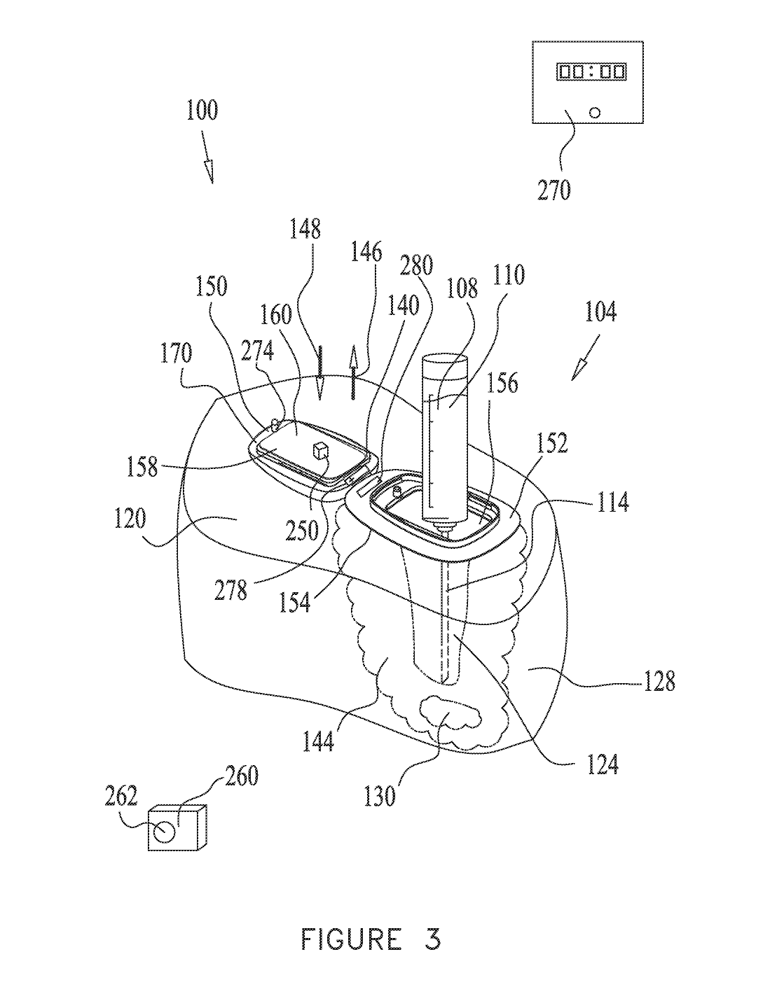

FIGS. 3-7 illustrate exemplary systems 100 for regulating the absorption of a drug in the body of a patient, according to some embodiments of the present disclosure. The system 100 may include a drug delivery device 104 comprising a drug reservoir 108 for containing a substance, chemical and/or drug 110.

In some embodiments, the drug 110 may include a long-acting drug including a drug configured to affect the body over an extended time period. Generally a dose of the long-acting drug is disposed within the body, such as within a drug depot and is released therein during the extended time period.

The extended time period may include any one of: a time period of at least 8 hours or longer, a time period of at least 24 hours or longer, a time period of at least 2 days or longer, a time period of at least 3 days or longer, a time period of at least 1 week or longer, a time period of at least a month or longer, a time period of a few months or longer. This extended time period may include the lifetime of the drug in the patient body.

Exemplary long-acting drugs may include a drug for affecting and/or controlling blood sugar, such as insulin. Examples of a long-acting insulin may include glargine insulin marketed under the trade name LANTUS®, a Lente insulin marketed under the trade name HUMULIN® and detemir insulin marketed under the trade name LEVEMIR®. An older version of insulin used for basal therapy is, for example, NPH (Neutral Protamine Hagedorn) insulin.

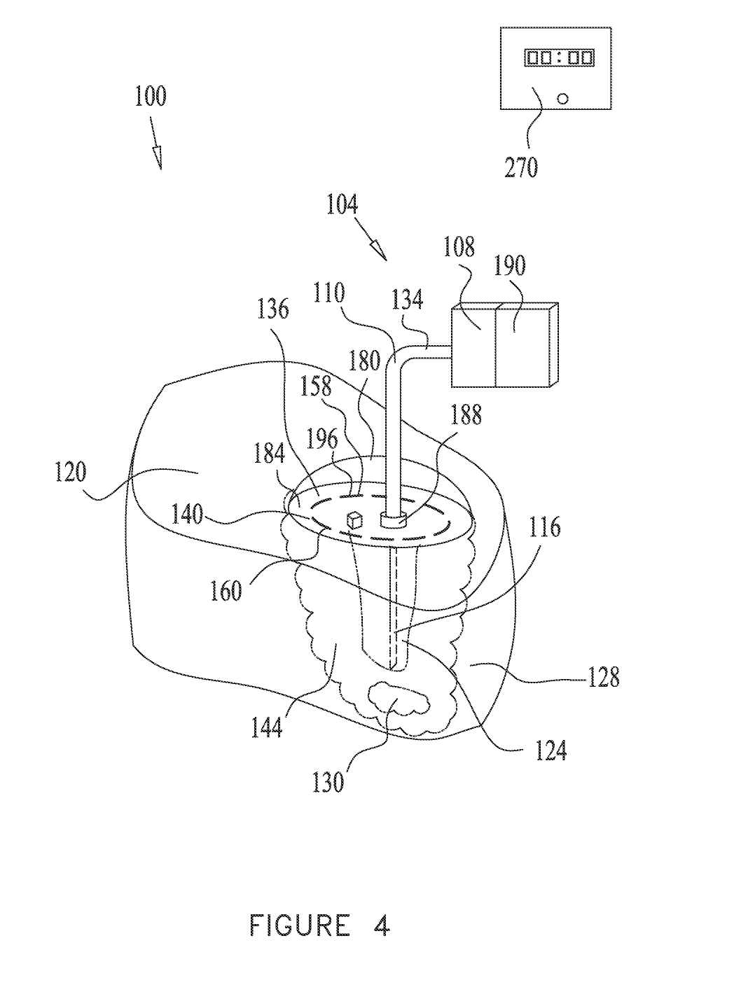

A needle 114 in FIG. 3 or a cannula 116 in FIG. 4, can deliver the drug 110 from the drug reservoir 108 through an outer surface of the skin 120 at a drug delivery site 124 to a tissue of the patient, such as the subcutaneous tissue layer 128.

In some embodiments the drug 110 may be administrated by injection where the drug 110 flows from the reservoir 108 through the needle 114 at the drug delivery site 124, including an injection site, to a drug depot 130.

In other embodiments, the drug 110 may be administrated by infusion where the cannula 116 (FIG. 4) can be inserted at the drug delivery site 124, including an infusion site. The drug 110 may be infused to the subcutaneous tissue layer 128 via a catheter 134.

The drug delivery site 128 may include any location in the body. The drug 110 may be delivered by any type of invasive drug administration, including any type of parenteral administration, which, for example, may include, but is not limited to, an intravenous administration or any type of injection or infusion such as, for example: subcutaneous, intradermal, transdermal, intramuscular, intraperitoneal, intrathecal, and/or the like.

According to some embodiments, upon drug delivery most of the long-acting drug resides at the drug depot 130 at an initial chemical structure. Upon a change in a property in the vicinity of the delivery site 124, the long-acting drug may transform its chemical structure and is perfused into the bloodstream of the patient at a predetermined absorption rate and/or a predetermined concentration.

The property may include any suitable property, such as pH level, blood perfusion, temperature and/or oxygen saturation, and/or the like in the vicinity of the delivery site 124 (and in the treatment area 144 described herein).

A substantial portion of long-acting drugs may reside at the drug depot 130 at the initial chemical structure formed of a precipitated material or a microprecipitated material. Upon a change in a property, such as the pH level in the vicinity of the delivery site 124, the long-acting drug may transform its chemical structure to other molecular structures, such as hexamers and then to dimmers and/or monomers, which may be perfused into the bloodstream of the patient at a predetermined absorption rate and/or a predetermined concentration. Such a long-acting drug may include the insulin glargine (LANTUS).

Some long-acting drugs may reside at the drug depot 130 at the initial chemical structure formed as an aggregate of hexamers. Upon a change in a property, such as the pH level in the vicinity of the delivery site 124, the long-acting drug may transform its chemical structure to dimmers or monomers and is perfused into the bloodstream of the patient at a predetermined absorption rate and/or a predetermined concentration. Such a long-acting drug may include the insulin detemir (LEVEMIR).

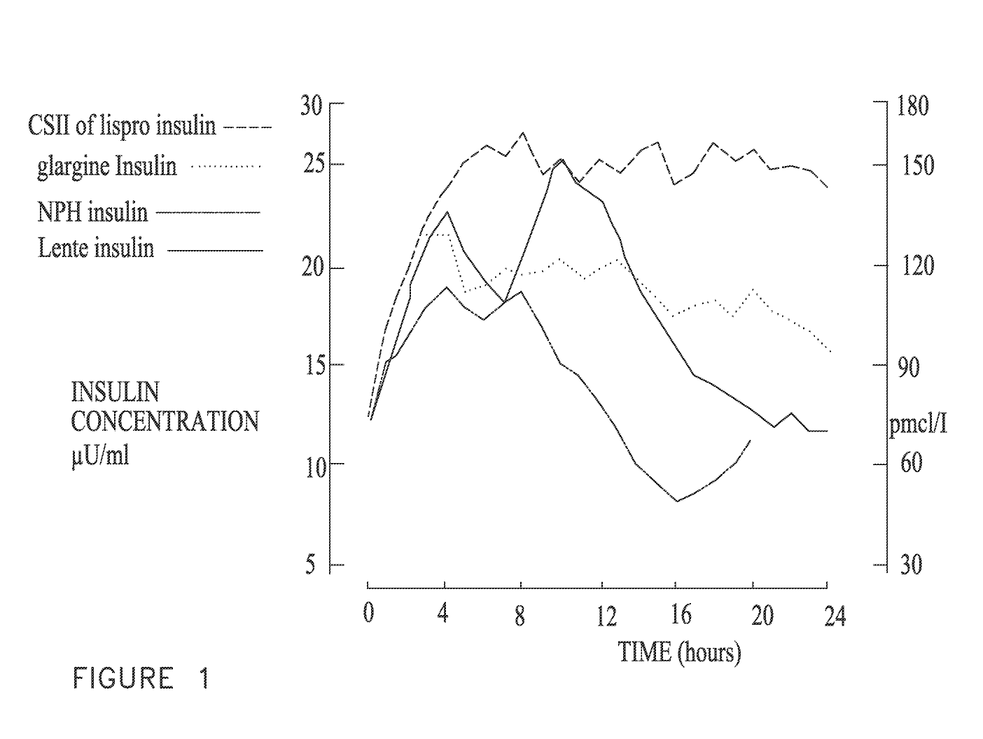

Ideally, the concentration of the long-acting drug in the bloodstream during its lifetime in the patient's body is to remain constant, thus resulting in a flat temporal profile. Yet, in reality there is variability in the concentration of the drug 110, resulting in a fluctuating temporal profile, as shown in FIG. 1.

A temporal profile may include the concentration of the drug 110 during a predetermined time period, such as during the lifetime of the drug in the patient body.

According to an embodiment of the disclosure, the system 100 may comprise a treatment element 140. The treatment element 140, through application of a treatment to a treatment area 144 is configured to modify the level of at least one property of the treatment area 144, thereby changing the absorption rate of the drug 110 perfused from the drug depot 130 to the bloodstream of the patient.

The treatment area 144 may surround and include the delivery site 124, wherein a substantial portion of the delivered drug 110 resides in tissue adjacent the treatment area 144 for an extended period of time and comprises the drug depot 130.

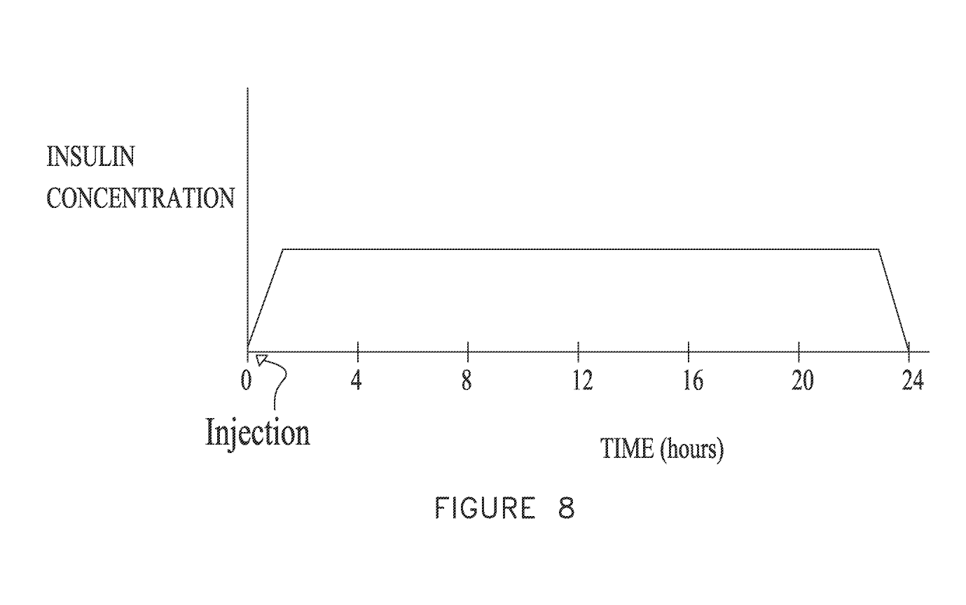

In some embodiments, the treatment element 140, through application of the treatment, may be configured to modify the absorption rate of the drug 110 from the drug depot 130 at an absorption rate designated for the release of a constant concentration of the drug 110. Thereby resulting in a generally flat temporal profile or a temporal profile with decreased fluctuations, such as shown in FIG. 8.

In some embodiments, the treatment element 140, through application of a treatment, is configured to modify the level of at least one property of the treatment area 144, thereby changing the drug's pharmacokinetic and/or pharmacodynamic profile. For example the drug's pharmacokinetic and/or pharmacodynamic profile may be smoothed, namely may have fewer fluctuations or may be substantially flat.

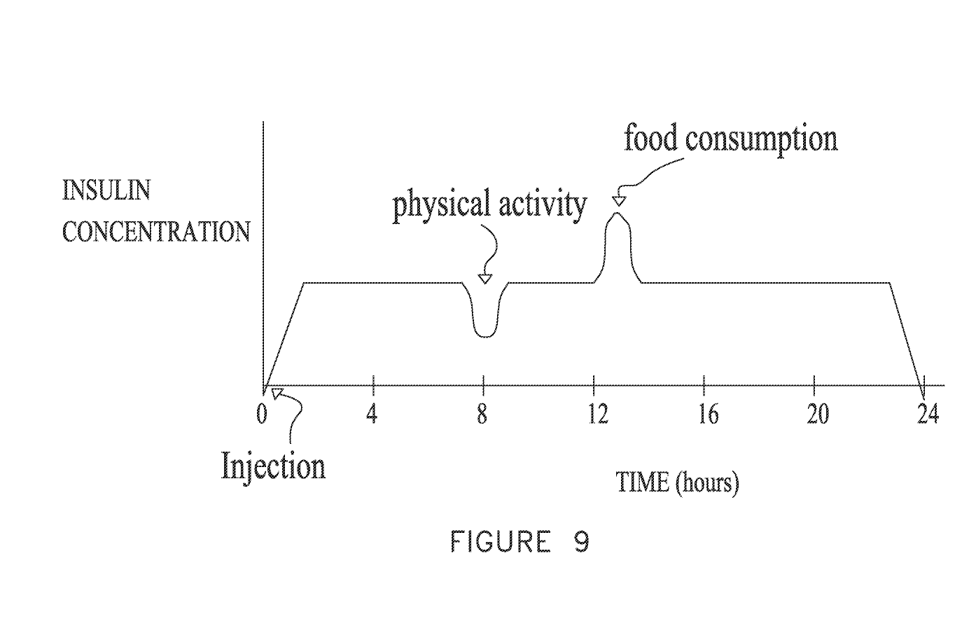

In some embodiments the treatment element 140, through application of the treatment, may be configured to adjust the drug absorption rate on-demand according to real-time measurements of the patient. For example, when the drug 110 comprises long-acting insulin, the applied treatment may be configured to adjust the drug absorption rate on-demand according to real-time blood glucose levels of the patient, thereby regulating the glucose level in a body of the patient. The treatment may be applied so as to regulate the glucose level of the patient and thus compensate for variability and fluctuations caused by the long-acting insulin, as well as fluctuations caused by the patient activity, such as eating, fasting and physical activity. For example, physical activity induces lower glycemic intake. Accordingly, the insulin levels in the patient's body should be decreased. Without applying the treatment, the long-acting insulin is unable to appropriately lower the insulin absorption rate. In some embodiments, the treatment may be applied to delay or decrease the insulin absorption rate, thereby providing the patient with the correct insulin absorption rate in real-time, as shown in FIG. 9. Additionally, upon applying the treatment to regulate the glucose level, only a single injection is required for a long time period such as for at least 8 hours, 24 hours, 1 day, a few days, a week a month or more.

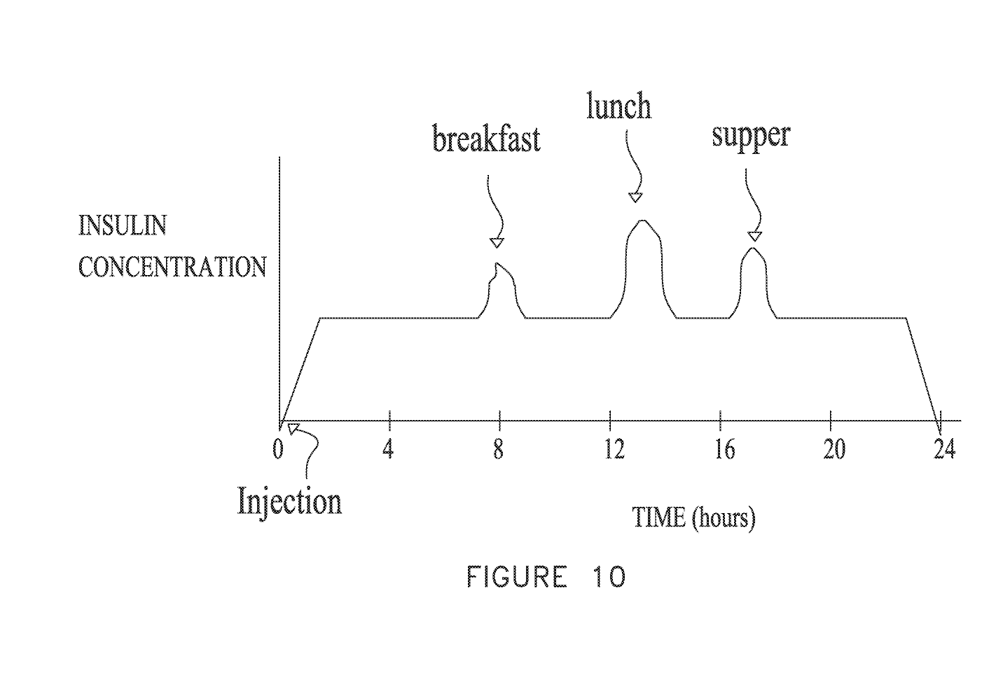

In some embodiments, the treatment may be configured to modify the property to effect a change in the absorption rate so as to mimic a short-acting drug. In some embodiments, the treatment may be configured to increase the property to affect a change in the absorption rate so as to mimic a bolus injection. In such a case a single injection of long-acting insulin will enable control of blood glucose levels both between meals and night time and around meals, as shown in FIG. 10. Accordingly, the patient applying the treatment for modifying the absorption rate of a long-acting drug, can eliminate or decrease the short-acting drug injections. For example, when the drug 110 includes insulin, a patient requiring basal-bolus therapy can administrate the long-acting insulin while eliminating or decreasing the administration of the short-acting insulin. Elimination or the decrease of the short-acting drug injections can be greatly advantageous for many reasons, such as limiting the occurrence of lipodystrophy, caused by repeated injections at an injection site. Lipodystrophy, besides indicating tissue damage at the injection site, may further induce an erratic, variable absorption rate of the injected drug the injection site.

In some embodiments, the treatment may be activated by the patient so as to increase the absorption of the long-acting insulin from the insulin depot to mimic the bolus dose injection, such that the mimicked insulin bolus injection achieves substantially the same pharmacokinetic and/or pharmacodynamics profile as a bolus dose injection.

The treatment element 140 may be placed at any suitable location. For example, the treatment element 140 may be placed on the skin 120 or in proximity thereto. In some embodiments, the treatment element 140 may be placed in proximity to the delivery site 124. In some embodiments, the treatment element 140 may be placed away from the delivery site 124. In some embodiments, the treatment element 140 may be placed on and/or around the treatment area 144. In some embodiments, the treatment element 140 may define the treatment area 144.

The treatment applied by the treatment element 140 may include, but not be limited to, for example, any one of: electrical, magnetic and/or mechanical stimulus, such as heating, cooling, mechanical vibrations, massaging, energy, acoustic stimulation (e.g. ultrasound), electromagnetic radiation, electric field stimulation, magnetic field stimulation, radio frequency irradiation, microwave irradiation, electrical stimulation, magnetic stimulation, Transcutaneous Electrical Nerve Stimulation (“TENS”), or the like, and/or any combination of the above treatments. In some embodiments, the treatment element 140 may stimulate or inhibit perfusion by introducing additional substances (in addition to the drug 110), for example, including, but not limited to, drugs, medicament, chemicals, biologically active bacteria, biologically inactive bacteria, a cream or emulsion, such as a cream with pH level modifying agents, or a cream with another property level modifying agent, or the like or also any combination of the above treatments.

In some embodiments, the pH level of the drug depot 130 changes by changing the temperature of the treatment area 144. In some embodiments, applying treatment by heating, may increase the pH level of the treatment area 144 and/or may increase blood perfusion in the treatment area 144. In some embodiment, applying treatment by cooling, may decrease the pH level of the treatment area 144 and/or may decrease blood perfusion in the treatment area 144.

In some embodiments, applying treatment by increasing the acidity of the pH level, such as less than a pH level of 7, may increase the absorption rate. Applying treatment by decreasing the acidity of the pH level, such as more than a pH level of 7.4, may decrease the absorption rate. In some embodiments, applying treatment by neutralizing the pH level, namely by raising the pH level from an acidic level (less than 6.8) to a neutralized level, such as in the range of between about 6.8 to 7.4, the absorption rate may be decreased. In some embodiments, neutralizing the pH level includes changing the pH level to approximately the pH level of the body of the patient. In some embodiments, applying treatment may include modifying the pH level to about 4.6.

In some embodiments, applying treatment by applying negative pressure, such as by suction, may draw fluids to the treatment area 144, thereby increasing the blood perfusion, which may increase the absorption rate of the drug 110.

In some embodiments, applying treatment by applying positive pressure, such as by depression, may withdraw fluids from the treatment area 144, thereby decreasing the blood perfusion, which may decrease the absorption rate of the drug 110.

In some embodiments, applying treatment by ultrasound may target the treatment area 144 and thereby modify the chemical structure of the drug 110, such as by altering the size of the precipitates or microprecipitates urging their dissolve into hexomers, such as when the drug 110 comprises the insulin glargine.

In some embodiments, applying treatment by optical means such as illuminating the treatment area at a wavelength, operative to induce changes in any one of: the chemical structure of the drug 110, or the pH level, the oxygen saturation, or any other property modifying the absorption rate. In some embodiments, the optical means may include low light laser therapy operative to change the oxygen saturation or any other property.

In some embodiments, the treatment may comprise an analgesic or a vasodilator or any form of treatment that leads to improved vasodilatation of the treatment area 144.

In some embodiments, the treatment may comprise applying negative pressure on the treatment area 144 by forcing the skin 120 upwards in the orientation of an arrow 146, such as by suction of the skin 120. The treatment may comprise applying positive pressure on the treatment area 140 by forcing the skin 120 downwards in the orientation of an arrow 148, such as by depression of the skin 120.

In some embodiments, the treatment may be any form of treatment that leads to property change of the treatment area 144, which may include a change in pH levels, temperature, blood perfusion chemical structure of the drug 110, and/or oxygen saturation.

In some embodiments a combination of treatments may be applied, such as a first treatment for modifying a first property and second treatment for modifying a second property. For example, at least one treatment, configured for modifying the blood perfusion at the treatment area 144, may be applied, and another treatment, configured for changing the chemical structure of the drug 110 at the drug depot 130, may be applied. The combination of treatments, such as the first treatment and the second treatment, may be applied in any suitable manner. For example, the first and second treatments may be applied simultaneously, or the first treatment may be applied before the second treatment or the second treatment may be applied before the first treatment.

For example, the blood perfusion may be modified by application of a treatment including heating or cooling or any other treatment configured for increasing or decreasing the blood perfusion at the treatment area 144. The chemical structure of the drug 110 may be modified by application of any treatment configured for changing the chemical structure of the drug 110 at the drug depot 130, such as applying a treatment including massage, or suction, for example, thereby inducing the dissolving of precipitated material into hexemers and thereafter to dimmers and/or monomers.

The treatment element 140 may comprise a device 150 comprising a first unit 152, which may comprise a lower surface having a biocompatible adhesive 154 for coupling the first unit 152 to the skin surface 120. The first unit 152 may be formed with an aperture 156 overlying the skin surface 120 and for allowing the needle 114 to be inserted therethrough into the subcutaneous tissue layer 128.

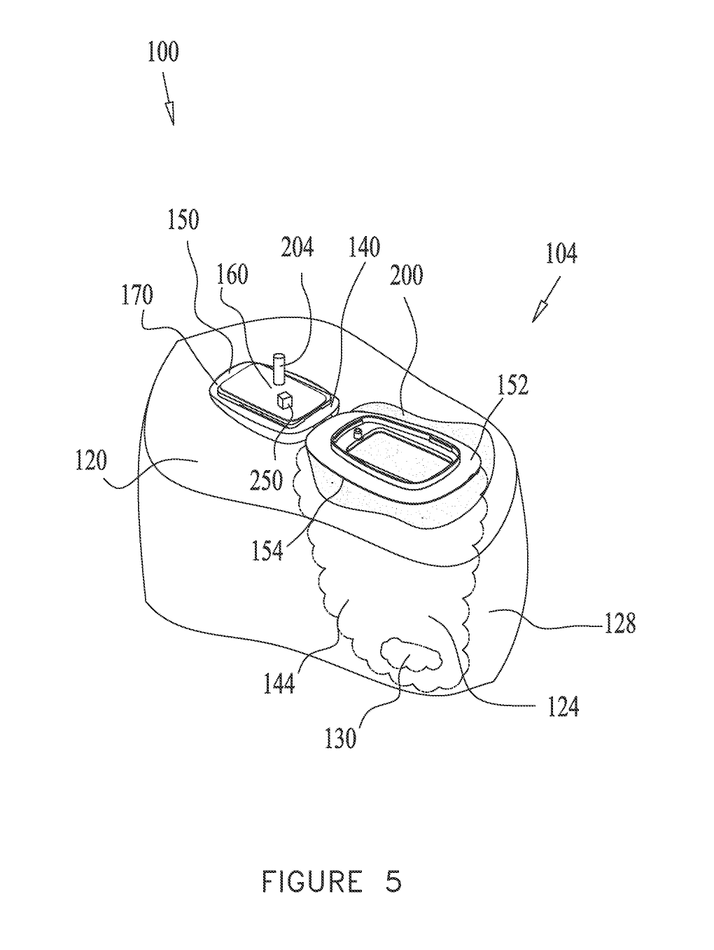

In some embodiments, the treatment element may comprise a treatment component 158 for applying the treatment, such as a heater or heating device (160 in FIGS. 3 and 196 in FIG. 4), a cooling device, a suction device, a transducer, a radiation delivery element, one or more electrodes, an injector, such as for injecting a cream, a dispenser (204 in FIG. 5), such as for dispensing a cream, and/or the like.

In the embodiment shown in FIG. 3, the treatment can be applied in a form of heat provided by a heating device 160. The heating device 160 may be applied to the skin surface 120 before, during and/or after the injection of the drug 110 is delivered. The device 150 may remain on the skin surface 120 for a selected time period.

The heating device 160 may be placed in any suitable location. In some embodiments, the heating device 160 can be embedded in a second unit 170, coupled to the first unit 152, as seen in FIG. 3. When the second unit 170 overlays the first unit 152, such that the device 150 is in a closed state, the heating device 160 overlies the skin 120, thus heating the treatment area 144. In some embodiments, the heating device 160 can be placed in the first unit 152 and then the second unit 170 can be obviated.

In some embodiments, the drug delivery device 104 can be configured as a syringe, as shown in FIG. 3. In some embodiments, the drug delivery device 104 can be configured as an injection pen.

In FIG. 4 the drug 110 is delivered by infusion. A device 180 may comprise the treatment element 140. The device 180 may comprise a lower surface comprising a biocompatible adhesive 184 for coupling the device 180 to the skin surface 120. The device 180 may be configured to be placed on the skin surface 120. The device 180 may comprise the catheter 134 formed on one end thereof, with the cannula 116, which can be inserted into the subcutaneous tissue layer 128.

In some embodiments, a connector 188 may connect the catheter 134 to the skin 120.

In some embodiments the catheter 134 may be connected at a second end thereof to the drug reservoir 108. In some embodiments, the device 180 may comprise an infusion pump 190, provided for control of the drug delivery from the drug reservoir 108. In other embodiments, the infusion pump 190 may be obviated.

The treatment element 140 may be placed in any suitable location. As seen in FIG. 4, the treatment element 140 may be configured in the device 180 and may be connected to the catheter 134. In some embodiments, the treatment element 140 may be disconnected from the catheter 134. In some embodiments, the treatment element 140 may be placed on the catheter 134 or in proximity thereto.

In a non-limiting example, such as shown in FIG. 4, the treatment can be applied in a form of heat provided by a heating device 196 within the device 180.

The heating device 196 may be applied to the skin surface 120 before, during and/or after the infusion of the drug 110 is administrated. The device 180 may remain on the skin surface 120 for a selected time period. In some embodiments, this selected time period can be prior to the infusion, during the infusion or a portion thereof and/or after the infusion is completed.

In some embodiments, the treatment element 140 may include a treatment device disclosed in any one of commonly owned International Patent Application Nos. PCT/IB2008/051044; PCT/IB2008/05104 6; PCT/IB2008/051049; PCT/IB2008/051050 PCT/IB2008/003547; PCT/IB2009/007600; PCT/IB2010/054476; PCT/IL2010/000623; PCT/IB2012/052335; PCT/IL2012/000211 the disclosures of which are incorporated herein by reference in their entireties.

In some embodiments, the treatment may be applied to increase or decrease the absorption rate of the drug 110, such as of a long-acting insulin. For example, the treatment may comprise heat, massage and/or applying negative pressure such as by suction and the absorption rate may increase thereby. The treatment may comprise cooling, applying positive pressure, such as by depression, and the absorption rate may decrease thereby.

In some embodiments, the treatment may be applied to modify the pH level of the treatment area 144. This may include increase in blood perfusion, which can result in a pH level increase. Similarly, decrease in blood perfusion can result in a pH level decrease. For example, the treatment may comprise heat, massage and/or applying negative pressure by suction and the blood perfusion rate may increase thereby. The treatment may comprise cooling, and/or applying positive pressure and the blood perfusion rate may decrease thereby.

FIG. 5 is an exemplary system 100. As seen in FIG. 5, the treatment element 140 may include a cream or an emulsion 200 comprising pH level modifying agents. For example, the modifying agents may increase the pH level in the treatment area 144. In some embodiments, an additional treatment element 140 may be provided for adjusting the permeation of the emulsion 200 into the treatment area 144. For example, the additional treatment element 140 may apply heat, massage or suction for increasing the permeation of the emulsion 200 into the treatment area 144, thereby increasing the pH level in the treatment area 144. In another example, the additional treatment element 140 may apply cooling, or depression for decreasing the permeation of the emulsion 200 into the treatment area 144, thereby decreasing the pH level in the treatment area 144.

In some embodiments, the treatment element 140 may include a cream or an emulsion 200 comprising temperature level or oxygen saturation level modifying agents. The additional treatment element 140 may be applied as described herein.

In some embodiments, the emulsion 200 may be embedded in the adhesive 154 and may be released therefrom by application of an additional treatment, such as by heat, pressure or massage. In some embodiments, the emulsion 200 may be dispensed by an injector or dispenser 204, such as shown for example in FIG. 5, wherein the emulsion is shown following dispensation from the injector or dispenser 204 onto the skin 120. The injector or dispenser 204 may be placed at any suitable location, such on the second unit 170. Upon closing the device 150 the second unit 170 may overlie the first unit 152 and the injector or dispenser 204 may dispense the emulsion 200 unto the skin 120.

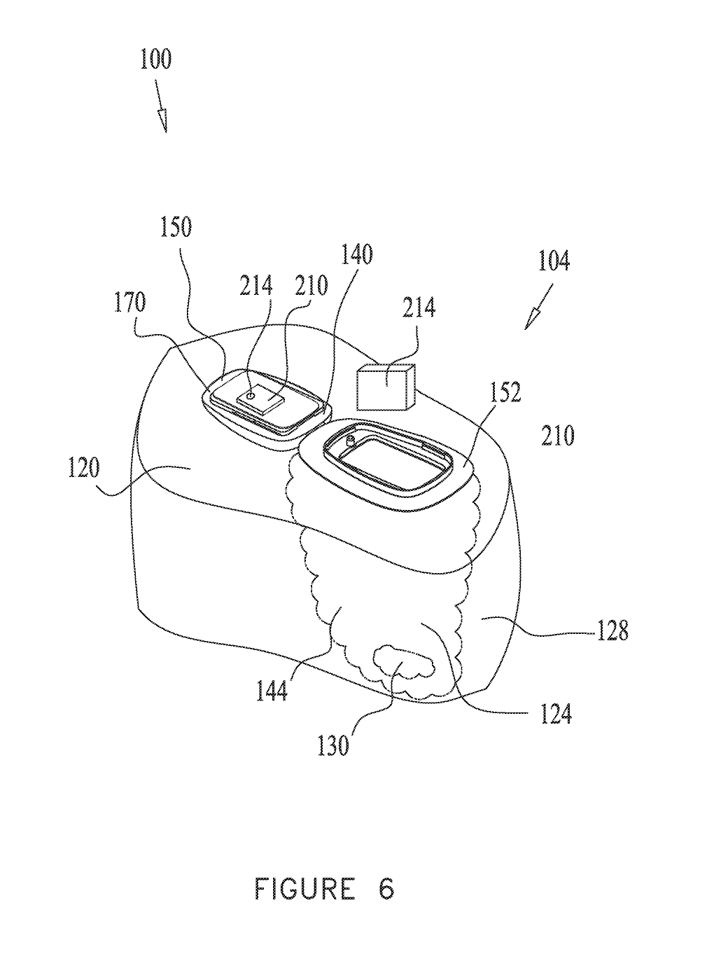

FIG. 6 is an exemplary system 100. As seen in FIG. 6, the treatment element 140 may include means for applying positive pressure on the skin 120 (which may be intermittent or constant, or a combination over certain time periods). For example, mechanical means such as a spring, a plurality of springs, and/or a pressing component 210 may be utilized for depressing the skin 120. The pressing component 210 may be pressed down for applying the positive pressure in any suitable manner, such as by positioning the second unit 170 on the first unit 152, thereby applying the positive pressure thereon in the orientation of arrow 148 (FIG. 3). The pressing component 210 may include a piston or any suitable components for applying positive (or negative) pressure. In some embodiments, application of the positive pressure may decrease the absorption rate of the drug 110 from the drug depot 130. For example, when the drug 110 comprises long-acting insulin, applying the positive pressure decreases the absorption rate from an insulin depot.

In some embodiments, electrical means may be utilized for applying the positive pressure or a combination of mechanical and electrical means, For example, a motor 214 or other electromagnetic means may be configured to activate the pressing component 210 and control the degree and the type of pressure (i.e. positive or negative) the pressing component 210 applies.

In some embodiments, the pressing component 210 may be elevated for applying the negative pressure on skin 120 in any suitable manner.

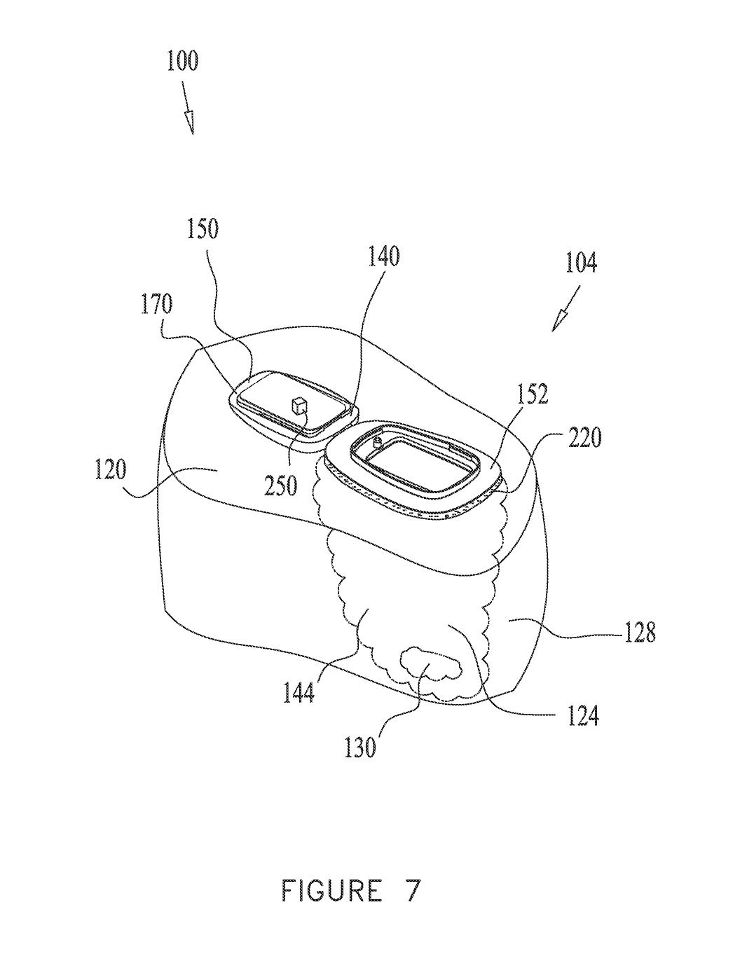

FIG. 7 is an exemplary system 100. As seen in FIG. 7, the treatment element 140 may include means for applying positive or negative pressure on the skin 120. For example, the adhesive 154 may be configured with a volume changing material 220 designed to expand and apply positive pressure in the orientation of arrow 148 (FIG. 3) or constrict upon induction of a stimulus. The stimulus may include a change in the electrical voltage of the adhesive 154 or any other component. Thereby, application of the positive pressure may decrease the absorption rate of the drug 110 from the drug depot 130.

The means for applying positive pressure on the skin 120, such as shown in FIGS. 6 and 7, may be modified according to the level of the property of the treatment area 144. This modification may include, for example, application of the positive pressure for decreasing the absorption rate of the drug 110. The applied positive pressure may be applied at a single pressure degree or the applied pressure may be varied for achieving a desired absorption rate. For increasing the absorption rate, the application of the positive pressure may be ceased. For further increasing the absorption rate, negative means may be applied on the skin 120, such as by suction, for example.

According to some embodiments, a sensor 250 may be provided and configured for detecting a property of the treatment area 144 and for providing a signal determinative of the property. The sensor 250 may be configured to determine a statistic or measurement associated the patient and/or the drug 110.

In some embodiments, a single sensor 250 may be used. In some embodiments a plurality of sensors 250 may be used for detecting different types of signals.

The statistic or measurement may include any one of the following: a time since last injection or drug delivery, blood glucose level, local blood perfusion, absorption rate, size of the drug depot 130, rate of change of the size of the drug depot 130, pH level of the treatment area 144 and/or temperature of the treatment area 144.

Based on the detected statistic or measurement the treatment may be applied to the surface of the skin 120 of the patient in an area surrounding and including the delivery site 124 (such as the treatment area 144). The treatment may be configured to modify the absorption of the drug 110 from the drug depot 130 into the bloodstream of the patient.

For example, the detected or determined property can be the pH level, the blood perfusion, the temperature oxygen saturation, the chemical structure of the drug 110 and/or the concentration of the drug 110 in the treatment area 144 and/or an amount of drug 110 remaining at the drug depot and/or the absorption rate of the drug 110 from the drug depot 130 and/or any indication of the pharmacokinetic and/or pharmacodynamics profile of the drug 110.

In some embodiments, sensor 250 may be configured to generate at least one signal determinative of the property of the drug 110 and generate a sensor signal representative thereof.

In some embodiments, the sensor 250 can be configured to measure the pH level at the treatment area 144. Based on the detected pH level the treatment element 140 may apply a treatment for modifying the pH level thereby modifying the absorption rate of the drug 110.

In some embodiments, the pH level may be detected by a sensor 250 configured with near infrared spectroscopy.

In some embodiments, the oxygen saturation may be measured by a sensor 250 including near infrared spectroscopy or by pulse oximetry, for example, or any other suitable method.

In some embodiments, the pH level may be detected indirectly by a sensor configured to measure the oxygen saturation and determining the pH level by application of the Bohr effect correlating between the hemoglobin's oxygen binding affinity and the pH level in the treatment area 144. In some embodiments, the pH level may be detected indirectly by a sensor configured to measure a carbon dioxide concentration in the treatment area 144 and determining the pH level by application of the Bohr effect correlating between the carbon dioxide concentration and the pH level in the treatment area 144

In some embodiments, the sensor 250 may comprise an optical sensor that measures optical properties of the skin surface 120, or a Laser Doppler Flowmeter (“LDF”) that can measure local blood perfusion in the treatment area 144.

In some embodiments, the sensor 250 may measure the size of the drug depot 130 or the rate of change of the size of the drug depot 130 so as to detect the amount of drug 110 yet to be released. The drug depot size may be measured in any suitable manner such as by employing imaging technology.

In some embodiments, the sensor 250 may be configured to measure the size of drug depot 130. Based on the measured drug depot size or rate of change thereof, the treatment element 140 may apply a treatment for modifying a property. thereby modifying the absorption rate of the drug 110.

In some embodiments, treatment may be applied when the rate of change in the size of the drug depot 130 decreases. Similarly, in some embodiments, the treatment may be stopped when the rate of change in the size of the drug depot 130 increases beyond a predetermined threshold. In some embodiments, a secondary treatment may be applied when the rate of change in the size of the drug depot 130 increases beyond a predetermined threshold. The secondary treatment may be applied to decrease the absorption rate or to stop release of the drug 110 from the drug depot 130. Such a secondary treatment may include cooling and/or depression, for example.

For example, wherein the drug 110 comprises a long-acting insulin, the treatment may be applied when the rate of change in the size of the insulin depot decreases. Similarly, in some embodiments, the treatment may be stopped when the rate of change in the size of the insulin depot increases beyond a predetermined threshold. In some embodiments, a secondary treatment is applied when the rate of change in the size of the insulin depot increases beyond a predetermined threshold.

In some embodiments, the drug depot size may be measured in any suitable manner, such as by electrical impedance measurement or optical measurements, for example. The sensor 250 may be configured to measure the size of the microprecipitates, drug molecule size, and/or concentration of the drug 110 at the drug depot 130 and/or in the treatment area 144.

In some embodiments, the size of the drug depot 130 may be detected by measuring microprecipitates, a concentration of drug molecules, and an amount of the drug molecules. For example, wherein the drug 110 comprises insulin, the size of the drug depot 130 may be detected by measuring microprecipitates, a concentration of insulin molecules, and an amount of insulin molecules.

In some embodiments, the sensor 250 can detect any information related to the drug 110, such as the dose, duration, frequency, flow rate and/or temperature of the drug 110. In some embodiments, the sensor 250 may detect when the drug 110 was delivered, such as when an injection has been made.

In some embodiments, the sensor 250 may be configured to detect a signal relating to patient activity, such as eating, fasting and/or physical activity. For example, the sensor 250 may include a movement tracking sensor, which may be used to detect the physical activity of a patient.

In some embodiments, the sensor 250 may comprise a glucose sensor for real-time measurements of the patient and a blood glucose monitor, which may be a continuous blood glucose sensor or a self-measurement blood glucose meter. In some embodiments, the treatment may be applied when the blood-glucose sensor or monitor detects a predetermined blood glucose level. In some embodiments, the treatment may be ceased when the blood-glucose monitor detects a predetermined glucose level. In some embodiments, the amount and/or type of treatment corresponds to a plurality of predetermined blood glucose levels. In some embodiments, the treatment may comprise heating the treatment area 144, when a first blood glucose level is detected. In some embodiments, the treatment may comprise cooling the treatment area 144, when a second blood glucose level, lower than the first blood glucose level, is detected.

In some embodiments, the sensor 250 may be configured to monitor the blood perfusion at or adjacent the insulin depot 130 or treatment area 144. Accordingly, treatment may be applied when the blood perfusion is equal or lower than a predetermined threshold. Of similarly, the treatment may be stopped when the blood perfusion is equal or greater than a predetermined threshold. The predetermined threshold may indicate a desired degree of blood perfusion, therefore when the blood perfusion is equal or lower than a predetermined threshold, the treatment may be applied so as to increase the absorption rate. When the blood perfusion is equal or greater than a predetermined threshold, the treatment may be stopped and/or a secondary treatment may be applied so as to decrease the absorption rate.

The sensor 250 or a plurality of sensors 250 may be placed at any suitable location, such as on the skin surface 120 and/or on any suitable location in proximity to the treatment area 144. In some embodiments, the sensors 250 may be embedded in the treatment element 140. In some embodiments, the sensors 250 may be placed on or adjacent the treatment element 140 or spaced away from the treatment element 140. In some embodiments, the sensors 250 may be placed on or adjacent the needle 114 or cannula 116 or spaced away from the needle 114 or cannula 116.

In some embodiments, the system 100 may comprise a controller 260 (FIG. 3). The controller 260 may comprise a processor 262. The controller 260 may be configured to apply the treatment by the treatment element 140. The processor 262 may include computer instructions operational thereon and configured for causing the processor 262 to operate the treatment element 140 to apply the treatment.

In some embodiments, the controller 260 may receive the sensor signal from the sensor 250 and can configure treatment by the treatment element 140 based on the detected property.

In some embodiments, the activation of the treatment by the controller 260 or any other means can be initiated in real time and “on-demand”. Additionally, the duration and intensity or degree of the treatment may be adjusted on-demand, in real-time. Adjusting the treatment may be controlled by the controller 260 and may be based upon a signal received from the sensor 250.

In some embodiments, the activation of the treatment by the controller 260 or any other means can be initiated before meal time or when a high blood glucose level is detected by the sensor 250 when configured for blood glucose measurement. This blood glucose measurement may be a self-blood glucose measurement conducted by the patient or a measurement obtained from a continuously detecting sensor 250 and automatically delivered to the controller 260. Using this treatment element, along with a continuous blood glucose sensor 250 and a controller 260 to control the operation of the system, may, for a closed loop system, automatically control blood glucose levels.

According to some embodiments, the treatment element may be activated remotely and/or automatically by the controller 260, according to data received from other components, such as from sensor(s) 250.

In some embodiments, the activation of the treatment may be predetermined and preprogrammed according to previously known data and/or the statistics or measurements. In some embodiments, the activation of the treatment may be according to a predetermined schedule, such in accordance with scheduled mealtime or physical activity, for example and/or according to a characteristic pharmacokinetics and pharmacodynamics profile of a drug.

In some embodiments, the activation of the treatment may be performed unrelated to delivery of the drug 110 by injection (FIG. 3) or infusion (FIG. 4). This feature is applicable to long-acting drugs 110 wherein many hours or even days pass from the delivery of the drug, yet the treatment is applied so as to modify the absorption rate of the long-acting drug 110.

In some embodiments, wherein the treatment element comprises the device 150, the treatment may be activated by manipulating the device 150, such as by positioning the device 150 in an open state, such a lifting the second unit 170 away from the first unit 152. Alternatively the activation may be controlled by a tinier 270 or remote device unrelated to the position of the device 152.

In some embodiments, the treatment element 140 may include an activation switch 274 for activating the treatment element. The switch 274 may be configured to increase or decrease the treatment. Additionally a display 278 may be provided to indicate a treatment degree, the property level or any other data required to apply the treatment.

In some embodiments, activation of treatment element 140, such as by the switch 274 and/or by any other mechanism/process, may be performed by the patient. In some embodiments activation of treatment element 140 may be performed automatically.

In accordance with some embodiments, a system for regulating the absorption of a long-acting drug in the body of a patient may include the system 100 and may comprise the treatment element 140 configured to apply treatment to the surface of the skin 120 surrounding and including the delivery site 124 where a dose of a long-acting drug is delivered into subcutaneous tissue 128.

The subcutaneous tissue adjacent and including the delivery site may comprises a long-acting drug depot 130 for containing the injected long-acting drug for an extended period of time. The treatment may be configured to modify the level of at least one property of the treatment area 144 so as to affect a change in the absorption rate of the long-acting drug from the drug depot.

The processor 262 having computer instructions operational thereon may be configured for causing the processor 262 to operate the treatment element 140 to apply treatment.

It is noted that all the features shown in FIGS. 5-7 can be used in an infusion system, such as shown in FIG. 4 or in any other drug delivery system.

According to an embodiment, the treatment may be applied and adjusted according to a protocol designed to ensure a desired, predetermined temporal profile of the drug concentration by modifying the drug absorption rate by applying a treatment by the treatment element 140.

In some embodiments, the desired predetermined profile is generally flat or has decreased fluctuations, such as shown in FIG. 8. Accordingly, the treatment protocols may be designed according to the type of drug, patient measurements, statistics or any other suitable factor.

For a temporal profile that is generally flat or has decreased fluctuations, an exemplary treatment protocol may be initiated at times where it is known that the insulin concentration in the blood is low, for example 1-2 hours after injection or few hours before a new injection would be given.

An additional exemplary treatment protocol may include activating the treatment element to achieve a flat concentration profile of the long acting drug in the blood, as shown in FIG. 8. It is known that without applying the treatment, the temporal profile of insulin glargine (LANTUS) is not flat during the 24 h period between injections. The concentration is lower during the first 3-4 hours after injection and 3-4 hours before the next injection. A treatment protocol may include treatment activation during the first and last 4 hours during the 24 hours period. The treatment can be applied intermittingly for 10 minutes with a 10 minutes interval therebetween. The treatment protocol can include warming the site to change local blood flow and tissue pH level or mechanical massage of the injection site to increase the absorption rate to accelerate release of the insulin from the precipitated material to the blood system.

As seen in FIG. 8, following a long-acting insulin injection at the commencement of a 24 hour period, at T=“0”, the concentration profile remains substantially flat during the 24 hour period, until a new injection is required.

For a temporal profile that modifies the drug release on-demand, for regulating the glucose level in a body of the patient, as shown in FIG. 9, an exemplary treatment protocol includes: cooling the treatment area to a temperature of 30° C. and depressing the skin of the treatment area, for delaying the insulin release (e.g. a long-acting insulin analog), 30 minutes prior to a planned physical activity session. The treatment for delaying the insulin absorption rate may be applied for a period of 10 minutes at intervals of 10 minutes, during the physical activity period. The duration of the physical activity session can be provided by the user or may be provided via data generated by a signal from a movement tracking sensor. An additional treatment may include heating the treatment area to a temperature of 40° C. and applying suction or massage to the skin of the treatment area, initiated, for example, 20 minutes prior to commencement of food consumption or along with the food consumption, for a period of 10 minutes, at intervals of 10 minutes, for a period of one hour. The treatment can be stopped based on blood glucose reading received automatically or manually from a blood glucose meter.

As seen in FIG. 9, following a long-acting insulin injection at the commencement of a 24 hour period, at T=“0”, the concentration profile remains substantially flat. Due to application of the treatment around the physical activity session, the insulin absorption rate is decreased. Due to application of the treatment around the food consumption, the insulin absorption rate is increased. in between the physical activity and food consumption, the concentration profile remains substantially flat. The treatment may be applied at various degrees according to a required insulin absorption rate.

For a temporal profile that mimics the short-acting drug at a bolus rate, as shown in FIG. 10, an exemplary treatment protocol including heating and/or application of suction may be initiated, for example, up to 30 minutes prior to commencement of a meal, for a period of 10 minutes at intervals of 10 minutes for a period of two hours.

As seen in FIG. 10 following a long-acting insulin injection at the commencement of a 24 hour period, at T=“0”, the concentration profile remains substantially flat. Due to application of the treatment around mealtime, the insulin absorption rate is increased at breakfast, lunch and supper. In between the mealtimes the concentration profile remains substantially flat. The treatment may be applied at various degrees according to a required insulin absorption rate. For example, it is seen that during lunch time in the specific example shown in FIG. 10, a bigger meal is consumed and accordingly a larger amount of insulin needs to be absorbed at an increased rate in comparison with breakfast, wherein a smaller meal is consumed and accordingly a smaller amount of insulin needs to be absorbed.

In some embodiments, upon detecting an amount of the drug 110, such as the long-acting drug in the drug depot 130 is below a predetermined threshold, an alert may be generated. The alert may be at least one of: sounding an acoustic noise, vibrating, lighting an indicator light, changing a color of the indicator light, and sending an alert to the patient via at least one of: an SMS message, a text message, an MMS message, an email, and a phone call. In some embodiments, an intensity of the alert may change depending upon the amount of the drug 110 in the drug depot 130.

The system may further include a sensor to detect that an amount of the long-acting drug in the drug depot is below a predetermined threshold, and an alerting mechanism 280 (FIG. 3) to generate an alert.

The alert may be at least one of: sounding an acoustic noise, vibrating, lighting an indicator light, changing a color of the indicator light, and sending an alert to the patient via. at least one of an SMS message, a text message, an MMS message, an email, an alert provided by a mobile device application, and a phone call.

An intensity of the alert may change depending upon the amount of the long-acting drug in the drug depot.

Though many features described herein were described in relation to insulin and glucose regulation, the method and system 100 can be applicable to other drugs. In some embodiments, the method and system 100 described herein may be applied to a drug 110 comprising any other long-acting drugs. Some of the long-acting drugs benefitting from the method and system 100 may include the following non-limiting examples.

In some embodiments, the drug 110 may include any long-acting drug delivered by injection. By applying the treatment as described herein, the duration of the lifetime of the long-acting drug may be prolonged by injecting a larger dosage than would have been injected without the treatment. By applying the treatment, the absorption rate may be modified to release the drug for the prolonged duration. For example, a long-acting drug scheduled to be injected with a dosage sufficient for a 24 hour release period, can now be injected with a double dosage and the release may be slowed to last for a 48 hour period by application of the treatment.

In some embodiments, the drug 110 may include contraceptive medications. The contraceptive medication may be injected for a long period, such as a few months. The treatment element may modify the absorption rate according to the changes in the subject's body. For example, the sensor 250 may detect ovulation signs, such as the rise in the body's basal temperature or changes in amount and consistency of cervical mucus, and treatment may accordingly induce release of the contraceptive medication or increase the absorption rate of the contraceptive medication.

In some embodiments the drug 110 may include antipsychotic contraceptive medications.

In some embodiments, the drug 110 may include cancer treatment, such as long-acting cancer treatment for treating prostate cancer or treatment of pancreatic cancer with the long-acting somatostatin analogue lanreotide.

In some embodiments, the drug 110 may include long-acting pain relievers. In some embodiments, the drug 110 may include long-acting HIV medication. In some embodiments, the drug 110 may include asthma medication containing a bronchodilator maintained in the drug depot 130 and released into the blood system by the application of the treatment during asthmatic exacerbation.

In some embodiments, the drug 110 may include long-acting multiple sclerosis treatment, such as COPAXONE®. By application of the treatment, a large dosage may be injected and released for a prolong time period. Thus the number of required injections may be reduced, thus significantly decreasing susceptibility to lipodystrophy.

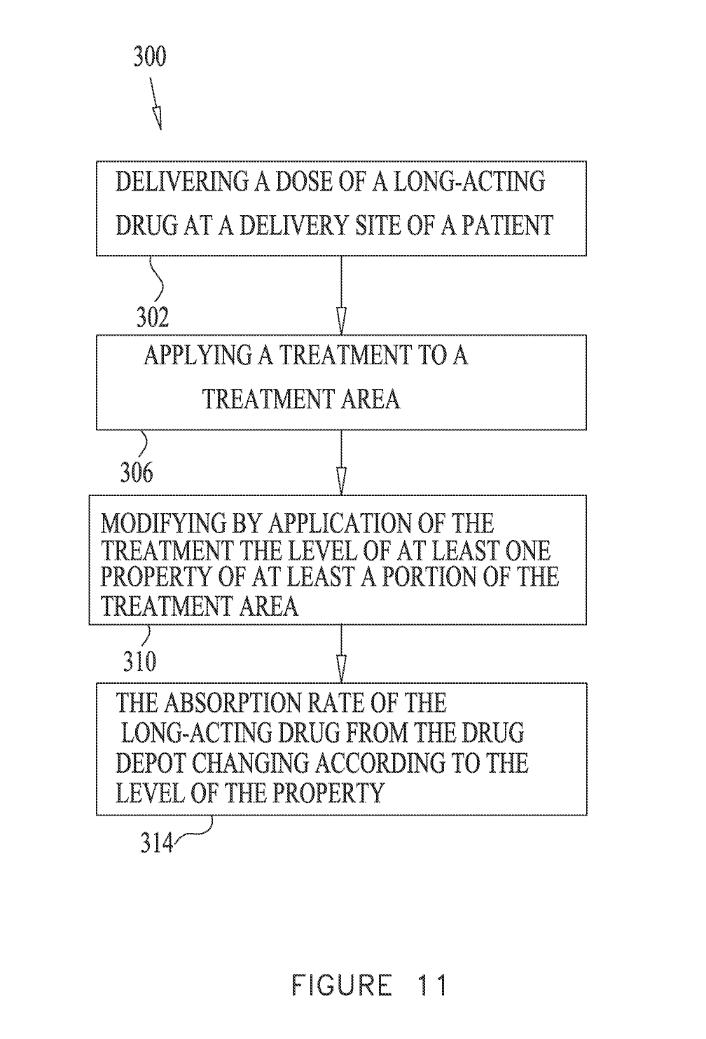

FIG. 11 is an exemplary schematic flow chart of a system and method 300 for regulating the absorption of a drag 110 in the body of a patient.

In some embodiments, a dose of a long-acting drug at a delivery site 124 of a patient may be delivered in any suitable manner 302, A treatment may be applied, 306, to a treatment area 144. The treatment area 144 may surround and include the delivery site 124, wherein a substantial portion of the delivered drug 110 resides in tissue adjacent the treatment area for an extended period of time and comprises a drug depot 130.

The treatment may be applied at any suitable time around the time of the drug delivery and/or unrelated to time the drug was delivered. For example, the treatment may be applied shortly before the drug delivery, a significantly long time before the drug delivery, during the drug delivery, a short time after the drug delivery, or a significantly long time after the drug delivery.

Application of the treatment the level of at least one property of at least a portion of the treatment area may be modified 310. The the absorption rate of the long-acting drug 110 from the drug depot 130 changes according to the level of the property 314.

Communication between the sensor 250, the controller 260, the processor 262 and any other components of the treatment element 140 or a component of the system 100 can be provided in any suitable manner. In some embodiments, the communication can be wired and provided through electrical connections. In some embodiments, the communication can be wireless via an analog short range communication mode, or a digital communication mode including WWI or BLUETOOTH®. Additional examples of such communication can include a network. The network can include a local area network (“LAN”), a wide area network (“WAN”), or a global network, for example. The network can be part of, and/or can include any suitable networking system, such as the Internet, for example, and/or an intranet.

Generally, the term “Internet” may refer to the worldwide collection of networks, gateways, routers, and computers that use Transmission Control Protocol/Internet Protocol (“TCP/IP”) and/or other packet based protocols to communicate therebetween.

In some embodiments the system 100 may comprise a single or plurality of transmission elements for communication between components thereof. In some embodiments, the transmission element can include at least one of the following: a wireless transponder, or a radio-frequency identification (“RFID”) device. The transmission element can include at least one of the following, for example: a transmitter, a transponder, an antenna, a transducer, and/or an RLC circuit or any suitable components for detecting, processing, storing and/or transmitting a signal, such as electrical circuitry, an analog-to digital (“A/D”) converter, and/or an electrical circuit for analog or digital short range communication.

In some embodiments, the controller 260 and/or any other relevant component of the system 100 can include a processor, a memory, a storage device, and an input/output device.

Various implementations of some of embodiments disclosed, in particular at least some of the processes discussed (or portions thereof), may be realized in digital electronic circuitry, integrated circuitry, specially configured ASICs (application specific integrated circuits), computer hardware, firmware, software, and/or combinations thereof. These various implementations, such as associated with the system 100 and the components thereof, for example, may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Such computer programs (also known as programs, software, software applications or code) include machine instructions/code for a programmable processor, for example, and may be implemented in a high-level procedural and/or object-oriented programming language, and/or in assembly/machine language. As used herein, the term “machine-readable medium” refers to any computer program product, apparatus and/or device (e.g., nontransitory mediums including, for example, magnetic discs, optical disks, flash memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a LCD (liquid crystal display) monitor and the like) for displaying information to the user and a keyboard and/or a pointing device (e.g., a mouse or a trackball, touchscreen) by which the user may provide input to the computer. For example, this program can be stored, executed and operated by the dispensing unit, remote control, PC, laptop, smartphone, media player or personal data assistant (“PDA”). Other kinds of devices may be used to provide for interaction with a user as well.

For example, feedback provided to the user may be any form of sensory feedback (e.g., visual feedback, auditory feedback, or tactile feedback), and input from the user may be received in any form, including acoustic, speech, or tactile input. Certain embodiments of the subject matter described herein may be implemented in a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components.

The components of the system may be interconnected by any form or medium of digital data communication (e.g., a communication network). Examples of communication networks include a local area network (“LAN”), a wide area network (“WAN”), and the Internet. The computing system according to some such embodiments described above may include clients and servers. A client and server are generally remote from each other and typically interact through a communication network. The relationship of client and server arises by virtue of computer programs running on the respective computers and having a. client-server relation to each other.

Any and all references to publications or other documents, including but not limited to, patents, patent applications, articles, webpages, books, etc., presented anywhere in the present application, are herein incorporated by reference in their entirety. Example embodiments of the devices, systems and methods have been described herein. As may be noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements/features from any other disclosed methods, systems, and devices, including any and all features corresponding to translocation control. In other words, features from one and/or another disclosed embodiment may be interchangeable with features from other disclosed embodiments, which, in turn, correspond to yet other embodiments. Furthermore, one or more features/elements of disclosed embodiments may be removed and still result in patentable subject matter (and thus, resulting in yet more embodiments of the subject disclosure). Still further, some embodiments are distinguishable from the prior art due to such embodiments specifically lacking one or more features which are found in the prior art. In other words, some embodiments of the disclosure include one or more negative limitations to specifically note that the claimed embodiment lacks at least one structure, element, and/or feature that is disclosed in the prior art.

1. (canceled)

2. A method for regulating the absorption of a long-acting drug in the body of a patient, comprising:
delivering a dose of a long-acting drug at a delivery site of a patient;
applying a treatment to a treatment area surrounding and including the delivery site, wherein a substantial portion of the delivered drug resides in tissue adjacent the treatment area for an extended period of time and comprises a drug depot;
wherein said treatment is configured to modify a level of at least one property of at least a portion of the treatment area; and
wherein an absorption rate of the long-acting drug from the drug depot changes according to the level of the property.

3. (canceled)

4. A system for regulating a glucose level in a body of a patient, comprising:
a treatment element configured to apply treatment to the surface of the skin surrounding and including an injection site where a dose of a long-acting insulin is injected into subcutaneous tissue,
wherein
the subcutaneous tissue adjacent and including the injection site comprises a long-acting insulin depot for containing the injected long-acting insulin for an extended period of time, and the treatment is configured to modify a level of at least one property of the treatment area so as to effect a change in an absorption rate of the long-acting insulin from the insulin depot;
a processor having computer instructions operational thereon configured for causing the processor to operate the treatment element to apply treatment.

5. The system of claim 4, wherein the treatment element comprises at least one of: a heater, a cooling device, a suction device, a transducer, a radiation delivery element, one or more electrodes, an injector, and a dispenser.

6. The system of claim 4, wherein the treatment comprises at least one of:
heating, cooling, suction, depression, massage, energy, radiation, mechanical vibration, electrical stimulation, acoustic stimulation, magnetic stimulation, electromagnetic stimulation, radio frequency irradiation, microwave irradiation, injection of an additional substance, application of a cream, Transcutaneous Electrical Nerve Stimulation (“TENS”), drugs, medicament, chemicals, biologically active bacteria, biologically inactive bacteria, an analgesic and a vasodilator.

7. The system of claim 4, wherein the at least one property of the treatment area comprises at least one of: temperature, pH, blood perfusion, chemical structure of the insulin and oxygen saturation.

8. The system of claim 4, wherein the absorption rate of the long-acting insulin is increased or decreased based upon the treatment applied by the treatment element.

9. (canceled)

10. The system of claim 7, wherein the computer instructions are additionally configured to cause the processor to operate the treatment element to cause at least one of:
a modification of the pH level of at least a portion of the treatment area; and
a change in the chemical structure of the long-acting insulin from at least any one of: a microprecipitated structure to a hexamer structure and a hexamer structure to a monomer structure.

11. The system of claim 4, wherein the computer instructions are additionally configured to cause the processor to operate the treatment based on an activity level of the patient.

12. The system of claim 4, wherein the computer instructions are additionally configured to cause the processor to operate the treatment element to modify the level of the at least one property so as to change the absorption rate to mimic a bolus dose injection.

13. (canceled)

14. (canceled)

15. The system of claim 4, wherein the treatment element is activated by the patient or based on computer instructions configured to cause the processor to operate the treatment element to apply treatment.

16. The system of claim 4, wherein the computer instructions configured to cause the processor to operate the treatment element to apply treatment based on a predetermined schedule.

17. The system of claim 4, further comprising a sensor to detect when an injection has been made.

18. The system of claim 4, further comprising a blood glucose monitor provided with the treatment element, wherein the blood glucose monitor is configured to detect a blood glucose level of the patient.

19. The system of claim 18, wherein the computer instructions are additionally configured to cause the processor to operate the treatment element to apply treatment when the blood-glucose monitor detects a pre-determined blood glucose level or to cause the processor to operate the treatment element to cease applying the treatment when the blood-glucose monitor detects the pre-determined glucose level.

20. The system of claim 18, wherein the computer instructions are additionally configured to cause the processor to operate the treatment element to apply a first treatment to the treatment area when a first blood glucose level is detected wherein the first treatment comprises at least heating the treatment area, and wherein the computer instructions are additionally configured to cause the processor to operate the treatment element to apply a second treatment to the treatment area when a second blood glucose level, lower than the first blood glucose level is detected, wherein the second treatment comprises at least cooling the treatment area.

21. (canceled)

22. (canceled)

23. The system of claim 22, further comprising a sensor configured to monitor blood perfusion at or adjacent the insulin depot and wherein the computer instructions are additionally configured to cause the processor to operate the treatment element to apply treatment when the blood perfusion is equal or lower than a predetermined threshold or to cause the processor to operate the treatment element to stop treatment when the blood perfusion is equal to or greater than the predetermined threshold.

24. (canceled)

25. The system of claim 4, further comprising a sensor to determine a size of the insulin depot or to determine a rate of change of the size of the insulin depot and wherein the computer instructions are additionally configured to cause the processor to operate the treatment element to apply treatment based upon the determined rate of change in the size of the insulin depot.

26. A system for regulating a glucose level in a body of a patient, comprising:
a processor to receive an indication of delivery of a long-acting drug into a drug depot of a patient, the drug depot comprising subcutaneous tissue adjacent and including the delivery site which retains a substantial amount of the delivered drug;
at least one sensor to detect at least one measurement associated with at least one of the patient and the drug;
a processor having computer instructions operational thereon configured to cause the processor to determine at least one treatment to apply to the surface of the skin of the patient in an area surrounding and including the delivery site, wherein the at least one treatment is configured to modify the absorption of the long-acting drug from the drug depot into the bloodstream of the patient based on the measurement; and
a treatment element to apply the at least one treatment, wherein the application of the at least one treatment element varies an absorption rate of the long-acting drug.

27. (canceled)

28. The system of claim 26, further comprising a sensor to detect an amount of physical activity of the patient, and wherein the computer instructions are additionally configured to vary the application of the at least one treatment based on the amount of physical activity of the patient.

29. (canceled)

30. The system of claim 26, further comprising:
a sensor to detect that an amount of the long-acting drug in the drug depot is below a predetermined threshold; and
an alerting mechanism to generate an alert.
